(19) United States
(12) Patent Application Publication
(10) Pub. No.: US 2024/0109056 A1
(43) Pub. Date: Apr. 4, 2024

(54) COMPOSITE IONIC LIQUID AND PREPARATION METHOD AND USE THEREOF

(71) Applicant: China University of Petroleum - Beijing, Beijing (CN)

(72) Inventors: Tao ZHENG, Beijing (CN); Rui ZHANG, Beijing (CN); Zhichang LIU, Beijing (CN); Haiyan LIU, Beijing (CN); Xianghai MENG, Beijing (CN); Chunming XU, Beijing (CN)

(21) Appl. No.: 18/156,979

(22) Filed: Jan. 19, 2023

(30) Foreign Application Priority Data
Sep. 28, 2022 (CN) 202211194807.4

(51) Int. Cl. B01J 27/24; B01J 23/10; B01J 27/10; B01J 37/04

(52) U.S. Cl. CPC B01J 27/24; B01J 23/10; B01J 27/10; B01J 37/04

(57) ABSTRACT

The present disclosure provides a composite ionic liquid and a preparation method and a use thereof. A first aspect of the present disclosure provides a preparation method of a composite ionic liquid, where an ammonium salt, a first metal salt, a second metal salt, and a third metal salt are sequentially added into a reactor for performing a reaction under different conditions, and the composite ionic liquid is obtained after the reaction is finished. The composite ionic liquid prepared by the method may be used as a catalyst to catalyze an alkylation reaction of isoparaffin with C4 olefin to obtain alkylated oil, which has the advantages of high catalytic activity, long catalytic life, low consumption, and better distribution of the resulting alkylated oil, etc, and thereby significantly reducing the production costs and improving the quality of the resulted alkylated oil.

### CROSS-REFERENCE TO RELATED APPLICATION

The present application claims priority to Chinese Patent Application No. 202211194807.4, entitled with “COMPOSITE IONIC LIQUID AND PREPARATION METHOD AND USE THEREOF”, filed with China National Intellectual Property Administration on Sep. 28, 2022. The disclosure of the aforementioned application is hereby incorporated by reference in its entirety.

### TECHNICAL FIELD

The present disclosure relates to a composite ionic liquid and a preparation method and use thereof, and, to the petrochemical technical field.

### BACKGROUND

With the rapid development of automotive industry and the gradual enhancement of human awareness of environmental protection, the market demand for low-carbon high-performance clean gasoline with low content of aromatics, low content of olefin, low vapor pressure and high octane number has been increasing. Alkylated oil has the advantages of high octane number and no sulfur, olefins and aromatics, and is an ideal blending component for clean gasoline.

Alkylated oil is a liquid product mainly of isooctane hydrocarbon obtained from alkane and olefin as raw materials through catalysis of catalyst, in which the catalyst is usually strong liquid acid such as concentrated sulfuric acid or hydrofluoric acid, but these strong liquid acids are extremely corrosive, and the development of environment-friendly catalysts has become a new research topic.

As an environment-friendly catalyst, ionic liquid has been widely used and studied in alkylation reaction catalysts, among them chloroaluminate ionic liquid has been applied to a certain extent due to its cost advantage. Chinese patent with a number of announcement of grant of patent right of CN 1184284C discloses a method for preparing alkylated oil agent through catalysis of butene and isobutane as raw materials by using an acidic ionic liquid as a catalyst, in which the proportion of C8 component in alkylated oil may reach 60-80%. A composite ionic liquid (CIL) with di-metal coordinated center structure may be synthesized by introducing weak Lewis-acidic transition-metal halide into a chloroaluminate ionic liquid. For example, Chinese patent with a number of announcement of grant of patent right of CN1203032C discloses a use of a composite ionic liquid as a catalyst to catalyze an alkylation reaction of isobutane and butene, in which the proportion of C8 in alkylated oil may reach 60-80%, and the proportion of a high octane number component, trimethylpentanes (TMPs), in C8, reaches above 70%, and the research octane number (RON), may reach 93-98.

Although composite ionic liquids have been relatively mature and widely used in terms of catalyzation of C4 raw materials, there are still side reactions such as cracking and oligomerization dominated by intermediate transition-state carbocations during alkylation reactions catalyzed by composite ionic liquids, thereby producing an acid-soluble oil (ASO) which affects the quality of alkylated oil and the activity of ionic liquids. Therefore, how to inhibit the generation of acid-soluble oil and improve the quality of alkylated oil and the catalytic activity of ionic liquids has become a technical problem to be urgently solved by those skilled in the art.

### SUMMARY

The present disclosure provides a composite ionic liquid and a preparation method thereof, for inhibiting the generation of acid-soluble oil and improving the quality of alkylated oil and the catalytic activity of the ionic liquid.

The present disclosure also provides a use of the above-mentioned composite ionic liquid in catalyzing an alkylation reaction.

The present disclosure also provides a preparation method of alkylated oil, which uses the above-mentioned composite ionic liquid as a catalyst.

A first aspect of the present disclosure provides a preparation method of a composite ionic liquid, the method including the following steps:
adding an ammonium salt into a reaction kettle under an inert gas atmosphere, adding a first metal salt at a controlled temperature of 50-80° C. then raising the temperature to 80-120° C. and performing a reaction for at least 2 hours to obtain a first mixture, where the ammonium salt is a hydrohalide of alkyl-containing amine, imidazole or pyridine, and the first metal salt is aluminum halide;
adding a second metal salt into the first mixture at a controlled temperature of 120-170° C., and obtaining a second mixture after the second metal salt dissolves completely in a reaction system, where the second metal salt is a halide, sulfate or nitrate of a second metal, and the second metal is one of copper, iron, zinc, gallium, nickel, cobalt, and platinum; and
adding a third metal salt into the second mixture at a controlled temperature of 120-170° C., and obtaining a composite ionic liquid after the third metal salt dissolves completely in the reaction system, where the third metal salt is a halide or nitrate of a third metal, and the third metal is a rare earth metal.

The present disclosure provides a preparation method of a composite ionic liquid, where an ammonium salt, a first metal salt, a second metal salt and a third metal salt are used as raw materials and are sequentially subjected to a reaction to obtain the composite ionic liquid, where the third metal salt is the rare earth metal salt. A multiple-metal site composite ionic liquid with aluminum-second metal-rare earth metal site and higher activity and stability is formed by dissolving the rare earth metal into the di-metal site composite ionic liquid, which helps to improve the hydrogen transfer activity of the composite ionic liquid catalyst, accelerate the reaction rate of isoparaffin and intermediate carbocations to generate high octane number C8 alkane through a hydrogen transfer reaction, promote the main alkylation reaction, effectively inhibit the occurrence of side reactions such as olefin polymerization and cyclization, reduce the generation amount of acid-soluble oil, and improve the quality of alkylated oil. In addition, the composite ionic liquid catalyst has better stability and longer catalytic life, and the raw material processing amount per unit mass of catalyst (1 kg) may reach 120 kg or more while maintaining high catalytic activity for complete conversion of olefin. Therefore, the composite ionic liquid prepared by the preparation method of the present disclosure has the characteristics of high catalytic activity, long catalytic life and low consumption, etc., and may be used to catalyze an alkylation reaction to improve the distribution of the resulted alkylation products, thereby improving the quality of alkylated oil and reducing the production costs.

In a specific embodiment, the preparation method specifically includes the following steps.

Step 1: an ammonium salt and a first metal salt are selected as raw materials to perform a first step reaction, where the ammonium salt is a hydrohalide of an alkyl-containing amine, imidazole or pyridine. Further, the ammonium salt is a hydrohalide of an alkyl-containing amine. The alkyl-containing amine refers to an amine whose molecular structure includes at least one nitrogen atom, and the nitrogen atom is saturated with four substituents, at least one substituent being an alkyl or benzyl. Further, the alkyl is C1-C6 alky, for example, the alkyl is one or more of methyl, ethyl, propyl, butyl, pentyl, and hexyl. Further, the alkyl is ethyl, and the ammonium salt is triethylammonium chloride (Et₃NHCl).

The first metal salt is aluminum halide, and further the first metal salt is aluminum chloride (AlCl₃).

A molar ratio of the ammonium salt to the first metal salt is 1:(1-2.5). Further, the molar ratio of the ammonium salt to the first metal salt is 1:(1.2-2.2). Further, the molar ratio of the ammonium salt to the first metal salt is 1:(1.6-1.8).

During the reaction process, a dry ammonium salt is added into a reaction kettle under an inert gas atmosphere. Since the reaction between solid metal salt and ammonium salt is rapid and exothermic, the temperature of the reaction system during the addition of the first metal salt should be kept within a certain range in order to prevent the temperature elevation from causing sublimation of the solid metal salt, i.e., the first metal salt is added when the temperature is controlled to be at 50-80° C. Specifically, the first metal salt may be added to the ammonium salt in at least two batches. It can be understood that the present disclosure is not limited to a batch-addition mode, and any method of adding metal salt for the purpose of controlling the heat generation and thus the temperature rise may be used appropriately.

After the first metal salt is completely added to the reactor kettle, the temperature of the reaction system is raised to 80-120° C. After the reaction is performed under stirring for at least 2 hours to completely transform the solid phase into a homogeneous liquid phase, thereby obtaining a first mixture.

Step 2: a second metal salt is selected as a raw material to perform a second step reaction of the first mixture with the second metal salt. Specifically, the second metal salt is a halide, sulfate or nitrate of a second metal, and the second metal is one of copper, iron, zinc, gallium, nickel, cobalt, and platinum, for example, a halide of copper, a nitrate of copper, a nitrate of zinc, nitrates of copper and zinc, etc. Further, the second metal salt is a chloride of the second metal, for example, cuprous chloride, copper chloride, ferrous chloride, etc. Further, the second metal salt is cuprous chloride, and adding the second metal salt may form a di-coordinated metal center anion including aluminum and a second metal.

A molar ratio of the ammonium salt to the second metal salt is 1:(0.1-2). Further, the molar ratio of the ammonium salt to the second metal salt is 1:(0.1-1). Still further, the molar ratio of the ammonium salt to the second metal salt is 1:(0.2-0.6).

During the reaction process, the second metal salt is also added in batches to prevent the metal sublimation caused by too fast reaction temperature rise. Specifically, the second metal salt is added to the first mixture when the reaction temperature is controlled to be 120-170° C. Further, the reaction temperature is controlled to be 125-160° C. Further, the reaction temperature is controlled to be 140-150° C. Under such temperature, the first metal salt is less liable to sublimation, thus not affecting the proportion of the ionic liquid; furthermore, such temperature helps to improve the solubility of the second metal salt, and after the second metal salt dissolves completely and disappears in the reaction system, a second mixture is obtained. The reaction temperature may be reasonably adjusted according to the type of the second metal salt.

Step 3: a third metal salt is selected as a raw material to perform a third step reaction of the second mixture with the third metal salt. Specifically, the third metal salt is a halide or nitrate of a third metal, and the third metal is a rare earth metal which is selected from one or more of scandium (Sc), yttrium (Y), lanthanum (La), cerium (Ce), praseodymium (Pr), neodymium (Nd), samarium (Sm), europium (Eu), gadolinium (Gd), terbium (Tb), dysprosium (Dy), holmium (Ho), erbium (Er), thulium (Tm), ytterbium (Yb), and lutetium (Lu). By adding one or more solid metal salts of rare earth element, a tri-coordinated metal center or multi-coordinated metal center anion including aluminum, the second metal and the third metal may be formed. Further, the third metal salt is a halide of the third metal: further, the third metal salt is a chloride of the third metal. Still further, the rare earth metal is selected from one or more of lanthanum (La), cerium (Ce), neodymium (Nd), samarium (Sm), and gadolinium (Gd), for example, the third metal is selected from one or more of lanthanum chloride (LaCl₃), cerium chloride (CeCl₃), and neodymium chloride (NdCl₃).

A molar ratio of the ammonium salt to the third metal salt is 1:(0.01-2). Further, the molar ratio of the ammonium salt to the third metal salt is 1:(0.01-1). Further, the molar ratio of the ammonium salt to the third metal salt is 1:(0.05-0.5). Still further, the molar ratio of the ammonium salt to the third metal salt is 1:(0.05-0.2).

During the reaction process, the third metal salt is added to the second mixture in batches at a controlled temperature of 120-170° C. under stirring. Further, the temperature is controlled to be 125-160° C.; still further, the temperature is controlled to be 140-160° C., and after the solid phase disappears completely in the reaction system, the composite ionic liquid is obtained.

Further, the third metal salt includes two or more rare earth metals. When the third metal includes at least two rare earth metals, a molar ratio of any one rare earth metal to the remaining rare earth metal is (0.05-50):1, and further (0.1-10):1. For example, when the third metal salt includes lanthanum chloride (LaCl₃) and cerium chloride (CeCl₃), a molar ratio of lanthanum chloride (LaCl₃) to cerium chloride (CeCl₃) is (0.05-50):1.

In addition, when the third metal salt includes two or more rare earth metals, the adding mode and sequence of different rare earth metal salts are not strictly limited under the condition of ensuring that no significant temperature rise occurs. For example, the rare earth metal salts may be added in sequence according to their types, or all the rare earth metal salts may be added after being mixed, or a certain parts of a first rare earth metal salt may be added, then another rare earth metal salt may be added, and finally the rest parts of the first rare earth metal may be added. For example, when the first rare earth metal salt is cerium chloride (CeCl₃) and a second rare earth metal salt is neodymium chloride (NdCl₃), cerium chloride (CeCl₃) is added firstly by parts in a batch-wise mode until all cerium chloride is added, and then neodymium chloride (NdCl₃) is added by parts in a batch-wise mode until all neodymium chloride is added. A reaction is performed under stirring at a temperature of 140-160° C. until a solid phase disappears completely; and after the reaction is over, the composite ionic liquid with a homogeneous phase is obtained by cooling the resulted solution.

In summary, the composite ionic liquid obtained by the above preparation method has the characteristics of high catalytic activity, long catalytic life and low consumption, etc., and may be used to catalyze an alkylation reaction to improve the distribution of the resulted alkylation products, thereby improving the quality of alkylated oil and reducing production costs.

A second aspect of the present disclosure provides a composite ionic liquid, prepared according to any one of the above mentioned preparation methods.

The composite ionic liquid provided by the present disclosure consists of cation and anion, where the cation is derived from the cation in the ammonium salt, and the anion is derived from the anion of multi-coordinated active center formed by the synergistic coupling of an aluminum salt, a second metal salt and a third metal salt. The multiple-metal site composite ionic liquid with aluminum-second metal-rare earth metal site and higher activity and stability is formed by dissolving a rare earth metal in a di-metal site composite ionic liquid, so that the composite ionic liquid provided by the present disclosure has the characteristics of high catalytic activity, long catalytic life and low consumption, etc., and may be used to catalyze an alkylation reaction to improve the distribution of the resulted alkylation products, thereby improving the quality of alkylated oil and reducing production costs.

A third aspect of the present disclosure provides a use of the above composite ionic liquid in catalyzing an alkylation reaction.

The composite ionic liquid provided by the present disclosure may be used as a catalyst to catalyze the alkylation reaction with isoparaffin and C4 olefin as raw materials to obtain alkylated oil, so that the yield of acid-soluble oil is less than 0.07% and the research octane number (RON) of the obtained alkylated oil may reach above 93.9. In addition, the composite ionic liquid catalyst also has better stability and longer service life, and the raw material processing amount per unit mass of catalyst (1 kg) may reach above 120 kg while maintaining high catalytic activity for complete conversion of olefin.

A fourth aspect of the present disclosure provides a preparation method of alkylated oil, the preparation method including the following steps:
performing an alkylation reaction of the above-mentioned reaction raw materials over the composite ionic liquid at a controlled temperature of 10-100° C. and a controlled pressure of 0.1-1.6 MPa for 0.5-60 minutes, and removing the composite ionic liquid to obtain the alkylated oil, where the reaction raw materials includes isoparaffin and C4 olefin.

In a specific embodiment, the isoparaffin is isobutane, the C4 olefin is one or more of 2-butene, isobutene and 1-butene, and further, the C4 olefin is isobutene and/or 2-butene.

The reaction conditions for catalyzing the alkylation reaction of isoparaffin and C4 olefin may be conventional in the art. For example, a molar ratio of isoparaffin to C4 olefin is (1-150):1. Further, the molar ratio of isoparaffin to C4 olefin is (10-100):1. When the C4 olefin includes isobutane and 2-butene, a molar ratio of isobutane to 2-butene is (10-20):1.

The reaction temperature needs to ensure that the composite ionic liquid under any composition is liquid, and the reaction pressure needs to ensure that the reaction material remains to be a liquid state. Specifically, the temperature of alkylation reaction is −20-100° C., further 0-25° C.; the absolute pressure of alkylation reaction is 0.1-1.6 MPa; and the reaction time of alkylation reaction may be 0.01-60 minutes, further 5-30 minutes.

The alkylation reaction may be performed in a reactor, and the reactor used may be a conventional reactor in the art, for example, a kettle reactor with a stirring device, a tubular reactor, a loop flow reactor with a plurality of feed injection points, a continuous production device used in industrial sulfuric acid or hydrofluoric acid alkylation reaction, and other continuous reaction device, etc. In any suitable alkylation reactor, the reaction raw materials may be in full contact with a multiple-metal site composite ionic liquid. The mixture of reaction raw materials may be in contact with the multiple-metal site composite ionic liquid in an intermittent process, a semi-continuous process or a continuous process.

Due to the relatively low affinity of the multiple-metal site ionic liquid for hydrocarbons such as reaction raw materials and alkylation products, as well as the relatively large density difference between the multiple-metal site ionic liquid and hydrocarbons, after the reaction is finished, the resulted solution after reaction may usually be separated into an upper phase (hydrocarbon-rich phase) mainly including alkylation products, and a lower phase mainly including the composite ionic liquid. The separation may be performed by using any other appropriate liquid-liquid separator. The liquid-liquid separator used may be a cyclone separator or a centrifuge separator.

The separated hydrocarbon-rich phase is processed and/or fractionated to obtain a fluid including at least alkylated oil and unreacted isoparaffin, while the separated composite ion liquid-rich phase is refluxed to the alkylation reactor.

In summary, the composite ionic liquid provided by the present disclosure may be used as a catalyst to catalyze the alkylation reaction of isoparaffin and C4 olefin as raw materials to obtain the alkylated oil, which makes the research octane number (RON) of the obtained alkylated oil reach above 93.9 and the yield of acid-soluble oil less than 0.07%. In addition, the composite ionic liquid catalyst also has better stability and longer catalytic life, and the raw material processing amount per unit mass of catalyst (1 kg) may reach above 120 kg while maintaining high catalytic activity for complete conversion of olefin. Therefore, the multiple-metal site composite ionic liquid obtained by the preparation method of the present disclosure has the advantages of high catalytic activity, long catalytic life, low consumption, better distribution of the resulting alkylation products, less generation of acid-soluble oil, etc., and may significantly reduce the production costs and improve the quality of alkylated oil when it is used to catalyze C4 alkylation reaction, thereby having good social and economic benefits, and being conducive to practical industrial application.

### DESCRIPTION OF EMBODIMENTS

To make the objectives, technical solutions, and advantages of the present disclosure clearer, the technical solutions in embodiments of the present disclosure will be described clearly and comprehensively below with reference to the embodiments of the present disclosure. Apparently, the described embodiments are merely a part rather than all embodiments of the present disclosure. All other embodiments obtained by persons of ordinary skill in the art based on embodiments of the present disclosure without creative effort shall fall within the protection scope of the present disclosure.

### Example 1

The method for preparing a composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 59.4 g (0.6 mol) of anhydrous CuCl into the first mixture in batches, heating up to 150° C., continuously stirring for 4 h to completely transform a solid phase into a liquid phase, and obtaining a second mixture;
Step 3: adding 12.32 g (0.05 mol) of anhydrous CeCl₃ into the second mixture in batches, heating up to 160° C., continuously stirring for 4 h to completely transform a solid phase into a liquid phase, cooling a mixture to room temperature, and obtaining a composite ionic liquid MIL-1.

### Example 2

The method for preparing a composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 49.5 g (0.5 mol) of anhydrous CuCl into the first mixture in batches, heating up to 150° C., continuously stirring for 4 h to completely transform a solid phase into a liquid phase, and obtaining a second mixture;
Step 3: adding 24.65 g (0.1 mol) of anhydrous CeCl₃ into the second mixture in batches, heating up to 160° C., continuously stirring for 4 h to completely transform a solid phase into a liquid phase, cooling a mixture to room temperature, and obtaining a composite ionic liquid MIL-2.

### Example 3

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 49.5 g (0.5 mol) of anhydrous CuCl into the first mixture in batches, heating up to 150° C., continuously stirring for 4 h to completely transform a solid phase into a liquid phase, and obtaining a second mixture;
Step 3: adding 12.32 g (0.05 mol) of anhydrous CeCl₃ into the second mixture in batches, maintaining stirring, adding 12.53 g (0.05 mol) of anhydrous NdCl₃ in batches, controlling the temperature to be 150° C., continuously stirring for 4 h at this temperature to completely transform a solid phase into a liquid phase, cooling a mixture to room temperature, and obtaining a composite ionic liquid MIL-3.

### Example 4

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 213.34 g (1.6 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 19.80 g (0.2 mol) of anhydrous CuCl into the first mixture in batches, heating up to 150° C., continuously stirring for 4 h to completely transform a solid phase into a liquid phase, and obtaining a second mixture;
Step 3: adding 12.26 g (0.05 mol) of anhydrous LaCl₃ into the second mixture in batches, maintaining stirring, controlling the temperature to be 150° C., continuously stirring for 4 h to completely transform a solid phase into a liquid phase, cooling a mixture to room temperature, and obtaining a composite ionic liquid MIL-4.

### Example 5

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 39.60 g (0.4 mol) of anhydrous CuCl into the first mixture in batches, heating up to 150° C., continuously stirring for 4 h to completely transform a solid phase into a liquid phase, and obtaining a second mixture;
Step 3: adding 36.97 g (0.15 mol) of anhydrous CeCl₃ into the second mixture in batches, heating up to 160° C., continuously stirring for 4 h to completely transform a solid phase into a liquid phase, cooling a mixture to room temperature, and obtaining a composite ionic liquid MIL-5.

### Example 6

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 19.80 g (0.2 mol) of anhydrous CuCl into the first mixture in batches, heating up to 150° C., continuously stirring for 4 h to completely transform a solid phase into a liquid phase, and obtaining a second mixture;
Step 3: adding 25.06 g (0.1 mol) of anhydrous NdCl₃ into the second mixture in batches, heating up to 150° C., continuously stirring for 4 h to completely transform a solid phase into a liquid phase, cooling a mixture to room temperature, and obtaining a composite ionic liquid MIL-6.

### Example 7

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 213.34 g (1.6 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 19.80 g (0.2 mol) of anhydrous CuCl into the first mixture in batches, maintaining stirring, heating up to 150° C., continuously stirring for 4 h to completely transform a solid phase into a liquid phase, and obtaining a second mixture;
Step 3: adding 24.65 g (0.1 mol) of anhydrous CeCl₃ into the second mixture in batches, maintaining stirring, adding 2.51 g (0.01 mol) of anhydrous NdCl₃ in batches, heating up to 160° C., continuously stirring for at least 4 h to completely transform a solid phase into a liquid phase, cooling a mixture to room temperature, and obtaining a composite ionic liquid MIL-7.

### Example 8

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 19.80 g (0.2 mol) of anhydrous CuCl into the first mixture in batches, heating up to 150° C., continuously stirring for 4 h to completely transform a solid phase into a liquid phase, and obtaining a second mixture;
Step 3: adding 13.45 g (0.05 mol) of anhydrous DyCl₃ into the second mixture in batches, heating up to 150° C., continuously stirring for 4 h to completely transform a solid phase into a liquid phase, cooling a mixture to room temperature, and obtaining a composite ionic liquid MIL-8.

### Example 9

The method for preparing the composite ionic liquid provided by this Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 225.19 g (1.9 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 9.90 g (0.1 mol) of anhydrous CuCl into the first mixture in batches, maintaining stirring, heating up to 150° C., continuously stirring for 4 h to completely transform a solid phase into a liquid phase, and obtaining a second mixture;
Step 3: adding 7.39 g (0.03 mol) of anhydrous CeCl₃ into the second mixture in batches, heating up to 160° C., continuously stirring for at least 4 h to completely transform a solid phase into a liquid phase, cooling a mixture to room temperature, and obtaining a composite ionic liquid MIL-9.

### Comparative Example 1

The method for preparing the composite ionic liquid provided by this Comparative Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 19.80 g (0.2 mol) of anhydrous CuCl into the first mixture in batches, maintaining stirring, heating up to 150° C., continuously stirring for 4 h to completely transform a solid phase into a liquid phase, cooling a mixture to room temperature, and obtaining a composite ionic liquid IL-1.

### Comparative Example 2

The method for preparing the composite ionic liquid provided by this Comparative Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 240.01 g (1.8 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 49.5 g (0.5 mol) of anhydrous CuCl into the first mixture in batches, maintaining stirring, heating up to 150° C., continuously stirring for 4 h to completely transform a solid phase into a liquid phase, cooling a mixture to room temperature, and obtaining a composite ionic liquid IL-2.

### Comparative Example 3

The method for preparing the composite ionic liquid provided by this Comparative Example included the following steps:
Step 1: weighing 137.65 g (1.0 mol) of dry [Et₃NH]Cl and adding it into a reaction kettle under nitrogen atmosphere, keeping a temperature to be 60-80° C., slowly adding 225.19 g (1.9 mol) of anhydrous AlCl₃ into the reaction kettle in batches, raising the temperature to 120° C. to perform a reaction, continuously stirring for 2 h to completely transform the solid into a homogeneous liquid phase, and obtaining a first mixture;
Step 2: adding 9.90 g (0.1 mol) of anhydrous CuCl into the first mixture in batches, maintaining stirring, heating up to 150° C., continuously stirring for 4 h to completely transform a solid phase into a liquid phase, cooling a mixture to room temperature, and obtaining a composite ionic liquid IL-3.

### Example 10

This Example provided a method for preparing an alkylated oil.

200 g of the composite ionic liquid MIL-1 was charged into a 500 mL autoclave and 100 mL of isobutane was then added. Nitrogen was introduced into the autoclave to maintain the pressure in the autoclave to be at 0.5 Mpa so as to keep reactants and products in a liquid phase.

The stirring speed was set to be 1500 r/min and the feed rate was set to be 700 mL/h. When the reaction temperature was 15° C., a double-plunger metering pump was turned on to continuously pump 2-butene (a molar ratio of isobutane to 2-butene was 10:1) into the autoclave.

Since the densities of alkylated oil and unreacted isobutane were less than that of the composite ionic liquid, after the reaction was finished, the composite ionic liquid was left in the autoclave and the alkylated oil and unreacted isobutane directly entered into an effluent collection tank.

### Example 11

The method for preparing alkylated oil provided by this Example may refer to Example 10, the difference being that the composite ionic liquid catalyst used was MIL-2.

### Example 12

The method for preparing alkylated oil provided by this Example may refer to Example 10, the difference being that the composite ionic liquid catalyst used was MIL-3.

### Example 13

The method for preparing alkylated oil provided by this Example may refer to Example 10, the difference being that the composite ionic liquid catalyst used was MIL-4.

### Example 14

The method for preparing alkylated oil provided by this Example may refer to Example 10, the difference being that the composite ionic liquid catalyst used was MIL-5.

### Example 15

The method for preparing alkylated oil provided by this Example may refer to Example 10, the difference being that the composite ionic liquid catalyst used was MIL-6.

### Example 16

The method for preparing alkylated oil provided by this Example may refer to Example 10, the difference being that the composite ionic liquid catalyst used was MIL-7.

### Example 17

The method for preparing alkylated oil provided by this Example may refer to Example 10, the difference being that the composite ionic liquid catalyst used was MIL-8.

### Example 18

The method for preparing alkylated oil provided by this Example may refer to Example 10, the difference being that the composite ionic liquid catalyst used was MIL-9.

### Comparative Example 4

The method for preparing alkylated oil provided by this Example may refer to Example 10, the difference being that the composite ionic liquid catalyst used was IL-1.

### Comparative Example 5

The method for preparing alkylated oil provided by this Example may refer to Example 10, the difference being that the composite ionic liquid catalyst used was IL-2.

### Comparative Example 6

The method for preparing alkylated oil provided by this Example may refer to Example 10, the difference being that the composite ionic liquid catalyst used was IL-3.

The organic phase products containing alkylated oil were qualitatively analyzed by gas chromatography-mass spectrometry to determine the composition of respective substances therein, and then the organic phase products were quantitatively analyzed by gas chromatography to determine the selectivity of products; and the methods for calculating the selectivity of products C5-C7, C8, C9 and trimethylpentanes (TMPs) and dimethylhexanes (DMHs), and the research octane number (RON) of alkylated oil were as follows:

1, Selectivity of C₅-C₇, C₈, C₉₊, trimethylpentanes (TMPs) and dimethylhexanes (DMHs):

$${Y}_{i} = \frac{Mass of component i in products}{Mass of alkylated oil} \times 100 \%$$

where Yᵢ was the selectivity of component i, and i represented one of C₅-C₇, C₈, C₉₊, trimethylpentanes (TMPs) and dimethylhexanes (DMHs).

2, Research Octane number (RON) of alkylated oil:

$$RON = {\sum }_{j = 1}^{n} {M}_{j} \times {RON}_{j}$$

where j represented each component in the alkylated product, Mⱼ was the volume percentage (%) of component j in the alkylated product, and RONⱼ was the research octane number (RON) of component j.

3, The single-pass catalyst life of the composite ionic liquid, represented by the raw material processing amount per unit mass of catalyst (kg of the raw material processing amount/kg of the catalyst), was specifically expressed by the processing amount of C4 raw materials when a certain amount of ionic liquid was added for continuous feeding until the ionic liquid began to deactivate (an olefin conversion rate of less than 99.9% was considered as the ionic liquid beginning to deactivate), in which there was no any active agent or regenerated ionic liquid added, and was calculated using the following formula:

Raw material processing amount per unit mass of catalyst (kg/kg)=Mass of processed raw material (kg)/Mass of catalyst (kg),
the olefin conversion rate was calculated according to the following formula:

$$Olefin conversion rate = \frac{Initial olefin mass - Olefin mass after reaction}{Initial olefin mass} \times 100 \% .$$

4, The amount of acid-soluble oil generated in the catalytic process of the composite ionic liquid was measured by a hydrolysis method, and was defined as the mass of acid-soluble oil generated by production of unit mass of alkylated oil (g of acid-soluble oil/1 kg of alkylated oil produced). The generation rate of acid-soluble oil was used to measure the generation status of acid-soluble oil and evaluate the inhibitory degree of catalyst to side reaction, and its calculation method was as follows: the generation rate of acid-soluble oil (%)=the generation amount of acid-soluble oil (g)/unit mass of alkylated oil (kg). The acid-soluble oil was extracted from a mixture of the ionic liquid and the acid-soluble oil by using the characteristic that the acid-soluble oil was insoluble in water while the multiple-metal site composite ionic liquid may be completely hydrolyzed, followed by centrifuging, drying, and etc., and weighing to obtain the mass of the obtained acid-soluble oil.

TABLE 1
Composition of the catalytic products of
Examples 10-18 and Comparative Examples 3-5

| | | | | | | | | | | Com- | Com- | Com- |
|---|---|---|---|---|---|---|---|---|---|---|---|---|
| | | | | | | | | | | parative | parative | parative |
| | Exam- | Exam- | Exam- | Exam- | Exam- | Exam- | Exam- | Exam- | Exam- | Exam- | Exam- | Exam- |
| Examples | ple 10 | płe 11 | ple 12 | ple 13 | ple 14 | ple 15 | ple 16 | ple 17 | ple 18 | ple 3 | ple 4 | ple 5 |
| Ionic Liquid | MIL-1 | MIL-2 | MIL-3 | MIL-4 | MIL-5 | MIL-6 | MIL-7 | MIL-8 | MIL-9 | IL-1 | IL-2 | IL-3 |
| Selectivity (%) | | | | | | | | | | | | |
| C₅ | 1.51 | 0.94 | 1.89 | 3.39 | 1.24 | 2.15 | 1.85 | 3.58 | 7.29 | 3.69 | 4.15 | 8.68 |
| C₆ | 1.83 | 0.57 | 1.38 | 2.41 | 1.21 | 1.68 | 1.58 | 2.01 | 3.77 | 1.74 | 2.53 | 4.56 |
| C₇ | 1.58 | 1.03 | 1.98 | 2.81 | 1.52 | 2.53 | 2.48 | 3.1 | 4.18 | 2.60 | 3.15 | 4.12 |
| C₈ | 90.99 | 95.96 | 90.92 | 87.31 | 93.21 | 87.12 | 87.36 | 86.29 | 78.48 | 86.78 | 82.72 | 76.18 |
| C₉₊ | 4.09 | 1.51 | 3.83 | 4.08 | 2.82 | 6.52 | 6.74 | 5.02 | 6.28 | 5.19 | 7.45 | 6.46 |
| Selectivity of | | | | | | | | | | | | |
| each component | | | | | | | | | | | | |
| in C₈ (%) | | | | | | | | | | | | |
| TMPs | 80.30 | 88.42 | 84.53 | 77.13 | 87.05 | 77.24 | 78.10 | 75.02 | 67.54 | 69.43 | 68.53 | 61.46 |
| 2,2,4-TMP | 53.54 | 55.36 | 43.28 | 49.87 | 44.82 | 41.62 | 40.33 | 45.65 | 40.06 | 47.15 | 44.75 | 34.99 |
| 2,3,4-TMP | 11.12 | 16.34 | 24.35 | 11.52 | 25.25 | 22.35 | 25.14 | 12.55 | 14.7 | 8.52 | 11.20 | 12.56 |
| 2,3,3-TMP | 15.65 | 16.73 | 16.90 | 15.74 | 16.98 | 13.27 | 12.74 | 16.82 | 12.78 | 13.76 | 12.58 | 13.91 |
| DMHs | 10.55 | 7.49 | 6.19 | 9.95 | 6.10 | 9.12 | 8.95 | 10.44 | 10.58 | 16.89 | 13.75 | 13.01 |
| 2,3-DMH | 1.52 | 1.55 | 2.01 | 1.57 | 2.12 | 1.97 | 3.59 | 2.01 | 3.07 | 1.80 | 1.66 | 2.02 |
| 2,4-DMH | 5.97 | 3.93 | 1.18 | 2.35 | 1.16 | 4.14 | 3.76 | 3.32 | 3.02 | 10.64 | 8.32 | 3.15 |
| 2,5-DMH | 2.69 | 1.70 | 2.87 | 5.82 | 2.69 | 2.79 | 1.39 | 4.86 | 4.22 | 3.94 | 3.34 | 7.14 |
| 3,4-DMH | 0.37 | 0.31 | 0.13 | 0.21 | 0.13 | 0.22 | 0.21 | 0.25 | 0.27 | 0.51 | 0.43 | 0.7 |
| TMPs/DMHs | 7.61 | 11.81 | 13.66 | 7.75 | 14.27 | 8.47 | 8.73 | 7.18 | 6.39 | 4.11 | 4.98 | 4.72 |
| Research Octane | 95.18 | 98.16 | 97.32 | 95.37 | 98.07 | 95.02 | 96.17 | 94.16 | 93.97 | 92.63 | 93.15 | 91.55 |
| Number (RON) | | | | | | | | | | | | |
| Acid-soluble oil | 0.50 | 0.12 | 0.32 | 0.41 | 0.11 | 0.56 | 0.51 | 0.58 | 0.67 | 1.26 | 1.03 | 1.30 |
| generation amount | | | | | | | | | | | | |
| (g/1 kg alkylated | | | | | | | | | | | | |
| oil) | | | | | | | | | | | | |
| Single-pass catalyst | 123.46 | 150.38 | 148.15 | 135.67 | 156.37 | 130.52 | 145.32 | 122.15 | 120.56 | 72.64 | 77.85 | 71.65 |
| life (kg of raw ma- | | | | | | | | | | | | |
| terial processing | | | | | | | | | | | | |
| amount/kg of cata- | | | | | | | | | | | | |
| lyst) | | | | | | | | | | | | |

It can be seen from the analysis results in Table 1:

(1) According to the comparison between Examples 10-18 and Comparative Examples 3-5, it can be seen that the composite ionic liquid catalyst provided by the present disclosure can make the yield of acid-soluble oil less than 0.07%, and the research octane number (RON) of the alkylated oil reach above 93.9; in addition, the composite ionic liquid catalyst also has better stability and longer service life, and the raw material processing amount per unit mass of catalyst (1 kg) may reach above 120 kg while maintaining high catalytic activity for complete conversion of olefin, which indicates that the addition of rare earth element has great influence on the yield and selectivity of the target product of the present disclosure.

(2) According to Examples 10-16 and Examples 17-18, it can be seen that further optimizing the type of the third metal salt and a molar ratio of the first metal salt, the second metal salt and the third metal salt helps to further improve the catalytic effect of the composite ionic liquid. Specifically, the yield of C8 component in the resulting alkylated oil was higher than 87 wt % and up to 95.96 wt %, the proportion of trimethylpentane in C8 component may reach 77-89%, the mass ratio of trimethylpentanes (TMPs) to dimethylhexanes (DMHs) was above 7.5 and up to 14.27, and the RON of the alkylated oil was above 95 and up to 98.16; and the mass of acid-soluble oil generated by production of unit mass (1 kg) of alkylated oil was only 0.11-0.56 g, the lowest being only 0.11 g.

Therefore, the composite ionic liquid provided by the present disclosure, as a catalyst for catalyzing an alkylation reaction of isoparaffin with C4 olefin, has excellent catalytic activity, longer catalytic life and good stability, so that the present disclosure have the advantages of low catalyst consumption, low production cost, etc., which is conducive to reducing production costs and is more suitable for continuous production.

Finally, it should be noted that the foregoing embodiments are merely intended for describing the technical solutions of the present disclosure rather than limiting the present disclosure; although the present disclosure is described in detail with reference to the foregoing embodiments, persons of ordinary skill in the art should understand that the technical solutions described in the foregoing embodiments may be modified or made equivalent substitutions to some or all technical features thereof, and these modifications and substitutions shall not cause the essence of the corresponding technical solutions to depart from the scope of the technical solutions of embodiments of the present disclosure.

What is claimed is:

1. A preparation method of a composite ionic liquid, wherein the method comprises the following steps:
adding an ammonium salt into a reaction kettle under an inert gas atmosphere, adding a first metal salt at a controlled temperature of 50-80° C., then raising the temperature to 80-120° C. and performing a reaction for at least 2 hours to obtain a first mixture, wherein the ammonium salt is a hydrohalide of alkyl-containing amine, imidazole or pyridine, and the first metal salt is aluminum halide;
adding a second metal salt into the first mixture at a controlled temperature of 120-170° C., and obtaining a second mixture after the second metal salt dissolves completely in a reaction system, wherein the second metal salt is a halide, sulfate or nitrate of a second metal, and the second metal is one of copper, iron, zinc, gallium, nickel, cobalt, and platinum; and
adding a third metal salt into the second mixture at a controlled temperature of 120-170° C., and obtaining a composite ionic liquid after the third metal salt dissolves completely in the reaction system, wherein the third metal salt is a halide or nitrate of a third metal, and the third metal is a rare earth metal.

2. The preparation method according to claim 1, wherein a molar ratio of the ammonium salt to the first metal salt is 1:(1-2.5); a molar ratio of the ammonium salt to the second metal salt is 1:(0.1-2); and a molar ratio of the ammonium salt to the third metal salt is 1:(0.01-2).

3. The preparation method according to claim 1, wherein the ammonium salt is triethylammonium chloride.

4. The preparation method according to claim 1, wherein the first metal salt is aluminum chloride.

5. The preparation method according to claim 1, wherein the second metal salt is a halide of copper.

6. The preparation method according to claim 1, wherein the third metal comprises one or more of lanthanum, cerium, neodymium, samarium, and gadolinium.

7. The preparation method according to claim 6, wherein when the third metal comprises at least two rare earth metals, a molar ratio of any one rare earth metal to the remaining rare earth metal is (0.05-50):1.

8. A composite ionic liquid, wherein the composite ionic liquid is prepared by the preparation method according to claim 1.

9. The composite ionic liquid according to claim 8, wherein a molar ratio of the ammonium salt to the first metal salt is 1:(1-2.5); a molar ratio of the ammonium salt to the second metal salt is 1:(0.1-2); and a molar ratio of the ammonium salt to the third metal salt is 1:(0.01-2).

10. The composite ionic liquid according to claim 8, wherein the ammonium salt is triethylammonium chloride.

11. The composite ionic liquid according to claim 8, wherein the first metal salt is aluminum chloride.

12. The composite ionic liquid according to claim 8, wherein the second metal salt is a halide of copper.

13. The composite ionic liquid according to claim 8, wherein the third metal comprises one or more of lanthanum, cerium, neodymium, samarium, and gadolinium.

14. The composite ionic liquid according to claim 13, wherein when the third metal comprises at least two rare earth metals, a molar ratio of any one rare earth metal to the remaining rare earth metal is (0.05-50):1.

15. Use of the composite ionic liquid according to claim 8 in catalyzing an alkylation reaction.

16. A preparation method of an alkylated oil, wherein the preparation method comprises the following steps:
performing an alkylation reaction of reaction raw materials over the composite ionic liquid according to claim 8 at a controlled temperature of −10-100° C. and a controlled pressure of 0.1-1.6 MPa for 0.01-60 minutes, and separating the composite ionic liquid to obtain alkylated oil, wherein the reaction raw material comprises isoparaffin and C4 olefin.
